(19) United States
(12) Patent Application Publication
(10) Pub. No.: US 2007/0166347 A1
(43) Pub. Date: Jul. 19, 2007

(54) Channeled endomural therapy

(75) Inventor: Marvin J. Slepian, Tucson, AZ (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Endoluminal Therapeutics, Inc.

(21) Appl. No.: 11/549,545

(22) Filed: Oct. 13, 2006

Related U.S. Application Data
(60) Provisional application No. 60/726,676, filed on Oct. 13, 2005.

(51) Int. Cl. A61K 35/12; A61M 25/10

(52) U.S. Cl. 424/423; 424/93.7; 604/95.03

(57) ABSTRACT

Diseases, aging, trauma, environmental exposure, infection and other events or agents can alter tissue function. In one embodiment, treatment is provided by therapeutically altering tissue function. This may be by continuing normal tissue function, suppressing tissue function, or enhancing tissue function. In a preferred embodiment, this treatment is effectuated by penetrating an organ, organ component or tissue structure and placing a supplemental material in a newly created space. This space is generally referred to herein as a “privileged space”, i.e. a space not otherwise present in native tissue. Supplemental materials can be deposited and secured within the zone. Supplemental materials include materials forming barriers, supports, and/or materials that deliver agents having a pharmacologic, biochemical, or physiologic effect in vivo. Suitable supplemental materials include polymeric and non-polymeric materials, pharmacologic agents, cells, tissue fragments, microorganisms, viral agents, or other reagents modifying tissue function. The supplemental material is typically in the form of a reservoir/depot, or continuous or discontinuous layer. In one embodiment, supplemental materials, and methods of use thereof, are provided for the continuous or discontinuous therapy of defined regions of an organ, organ component or tissue structure. The supplemental materials may be delivered directly to any one or more of the three zones, endoluminal (or ectomural, for solid organs), endomural, or ectoluminal, of organs, organ components, or tissue structures. The supplemental materials can include natural or synthetic polymeric materials that are biodegradable or non-biodegradable. The supplemental materials can also contain bioactive agents to effectuate a change in an organ or organ component in need thereof. For example, agents that result in a reduced/hyponormal response or an amplified/hypernormal response may be included in the supplemental materials.

### CROSS-REFERENCE TO RELATED APPLICATIONS

This application claims priority to U.S. Ser. No. 60/726,676, filed Oct. 13, 2005.

### FIELD OF THE INVENTION

This application relates to a discontinuous means of localized treatment of diseases and disorders in defined regions of an organ or organ component.

### BACKGROUND OF THE INVENTION

There are a number of organs or structures which have hollow or tubular geometry, for example blood vessels, such as arteries or veins, the gut and the bladder. These types of structures may be viewed as being organized with endoluminal, endomural, or ectoluminal regions and surfaces. In addition, many “solid” organ, such as the heart, liver, kidney and pancreas, possess true spaces such as cavities, cavernous sinuses, lumens, etc., which permit their characterization geographically or spatially in a similar fashion. Other solid organs may be viewed as containing two general regions, i.e., an ectomural zone and an endomural zone.

During the course of a lifetime, the function of an organ or organ component may change, including attainment of normal functions, loss of function (“hypo-normal function”), enhancement of function (“hyper-” or “supra-normal function”). Hypo-normal function may develop due to atrophy, toxemia, environmental exposure, infection, inflammation, malignancy, injury, ischemia, malnutrition, radiation exposure, temperature alteration, infiltrative processes, fibrotic processes, calcification, lipid insulation, atherosclerosis, and/or physical and/or mechanical stressors. Hypernormal function may develop due to hyperplasia, hypertrophy, meoplasia, different types of stimulation, including nutritional, metabolic, and/or supplement-stimulation, cellular infiltrative processes, exposure to a number of factors, including environment, radiation, hormones, temperature and/or pharmacological exposure, hyperemia, hyper- or super-fusion, malignancy, physical and/or mechanical stressors, and/or tissue implantation or transplantation.

In order to effectuate change in the organ or organ component, it may be necessary to locally alter, retard, or enhance function. Present therapies include conventional medication dosage forms (e.g. enteral or parenteral delivery of drugs), depot delivery, stent-based drug delivery, and polymeric endoluminal paving. However, there are limitations with each of these therapies. For example, with enteral or parenteral drug delivery, there is limited targeting of the organ or organ component of interest, which can cause undesired toxicity due to the exposure of the entire body to the drug. Depot delivery has similar limitations as enteral or parenteral drug delivery, in that there is limited targeting of the organ of interest and whole-body exposure to the drug delivered using the depot. Other limitations of depot delivery include toxicity or reactivity to the depot vehicle, limited delivery of novel therapeutics, and continuous (spatially and temporally) delivery from the depot site. The limitations of stent-based drug delivery include limited carrying capacity, single pharmacological release kinetics, limited areas of delivery, and delivery that is continuous along the body axis or components of the stent. The limitations of polymeric endoluminal paving include delivery that is continuous along or around a paving layer and largely confined to the endoluminal zone.

Therefore it is an object of the invention to provide materials, methods, and devices to effectuate change in an organ, organ component or tissue structure by local application or intervention thereby altering, retarding, or enhancing function of the organ, organ component, or tissue structures, or beyond.

### BRIEF SUMMARY OF THE INVENTION

Diseases, aging, trauma, environmental exposure, infection and other events or agents can alter tissue function. In one embodiment, treatment is provided by therapeutically altering tissue function. This may be by continuing normal tissue function, suppressing tissue function, or enhancing tissue function. In a preferred embodiment, this treatment is effectuated by penetrating an organ, organ component or tissue structure and placing a supplemental material in a newly created space. This space is generally referred to herein as a “privileged space”, i.e. a space not otherwise present in native tissue. Supplemental materials can be deposited and secured within the zone. Supplemental materials include materials forming barriers, supports, and/or materials that deliver agents having a pharmacologic, biochemical, or physiologic effect in vivo. Suitable supplemental materials include polymeric and non-polymeric materials, pharmacologic agents, cells, tissue fragments, microorganisms, viral agents, or other reagents modifying tissue function. The supplemental material is typically in the form of a reservoir/depot, or continuous or discontinuous layer.

In one embodiment, supplemental materials, and methods of use thereof, are provided for the continuous or discontinuous therapy of defined regions of an organ, organ component or tissue structure. The supplemental materials may be delivered directly to any one or more of the three zones, endoluminal (or ectomural, for solid organs), endomural, or ectoluminal, of organs, organ components, or tissue structures. The supplemental materials can include natural or synthetic polymeric materials that are biodegradable or non-biodegradable. The supplemental materials can also contain bioactive agents to effectuate a change in an organ or organ component in need thereof. For example, agents that result in a reduced/hyponormal response or an amplified/hypernormal response may be included in the supplemental materials.

### BRIEF DESCRIPTION OF THE DRAWINGS

FIG. 1A.1 is a diagram of a hollow organ 10 showing a channel 12 leading from the endoluminal zone 16 to the endomural zone 14. A “privileged space” 15 has been created by removing tissue from the endomural zone. FIG. 1A.2 is a diagram of a solid organ 30 showing a channel 32 leading from the ectomural zone 36 to the endomural zone 34. A “privileged space” 35 has been created by removing tissue from the endomural zone. FIGS. 1B-1D show delivery of supplemental materials in to various zones. FIG. 1B shows delivery of a material 20 at the tip of a device 18 through the connecting region 12 to the endomural zone 16. FIG. 1C shows delivery of a material 20 at the device 18 tip throughout the interior endomural zone 16 and channel or connecting region 12, i.e., within the endomural and endoluminal zones. FIG. 1D shows delivery of a material 20 throughout the endomural zone 16, connecting region 12, and exterior or ectoluminal zone 22.

FIG. 2 contains two diagrams; one diagram illustrates a continuous layer of a supplemental material on a tissue surface and the other diagram illustrates a discontinuous layer of a supplemental material on the surface of a tissue.

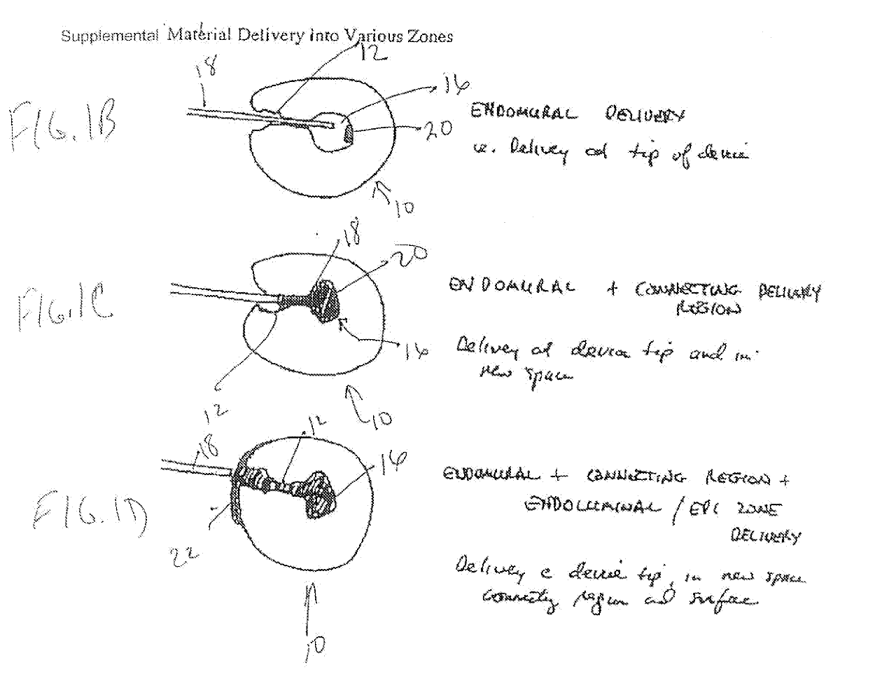

FIG. 3 is a diagram illustrating different locations of the effects achieved by administering a supplemental material containing a bioactive agent within one or more regions of an organ, organ component or tissue structure. “A” designates a local effect at a site where the bioactive agent is present. “B” designates an effect traversing tissue layers, throughout the area where the supplemental material is applied. “C” designates an effect within the tissue layer adjacent to the site where the bioactive agent is present within the supplemental material, achieved via secondary penetration into the tissue layer.

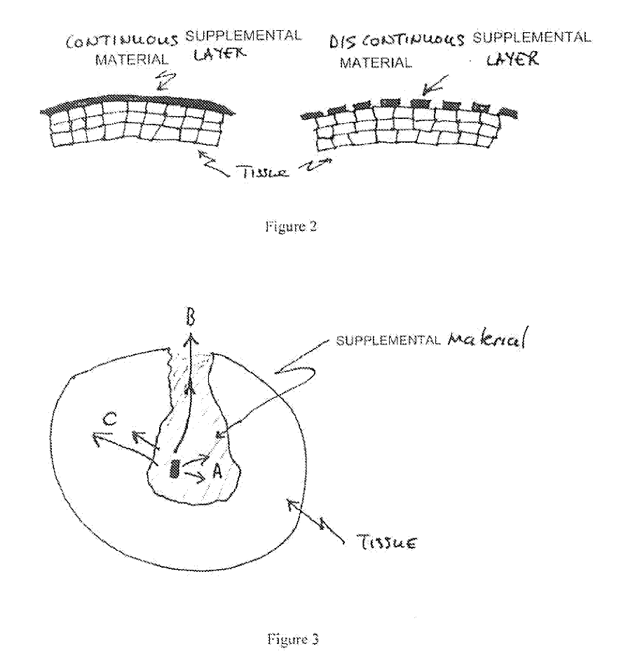

### DETAILED DESCRIPTION OF THE INVENTION

I. General Organization of Higher Animals:

The structural organization of higher animals such as mammals, including man, is that of multiple integrated and interactive tissue components. These tissues may be organized as discrete organs which are functional factories, e.g. liver producing biochemical mediators or device systems, e.g. heart—mechanically pumping blood and brain—electrically signaling and coordinating events. As referred to herein, “organs” include solid and hollow organs.

As generally used herein “tissue structure” means any collection of cells and matrix, including but not limited to tendons, ligamentous attachment, intervertebral discs, post traumatic adhesions, or other stromal or parenchymal components.

Animals also contain tissue components which are largely conduits for functional fluids such as blood, lymph, endocrine or exocrine secretions or gases. These tubular “organ components” or “conduits” are structures such as arteries, veins, lymphatics, bile ducts, ureters, fallopian tubes, etc.

a. Structure of Organs and Organ Components

Many “solid” organs, such as the heart, liver, kidney and pancreas, possess true spaces such as cavities, cavernous sinuses, lumens, etc. Organs containing true spaces may be generally described as having three regions or zones. These regions include: 1. the “ectoluminal” or outer zone (i.e. capsule, serosa, etc.), 2. the “endomural” or middle zone and 3. the “endoluminal” zone. If an organ is cut in cross-section the ectoluminal zone may be characterized as the outer 10%±10 cross-sectional area, the endomural zone as the mid 80%±10 and the endoluminal zone as the inner 10%±10.

In discrete organs the ectoluminal zone typically functions to protect and contain the organ. The endomural zone of the organ is typically the functional or “business end,” of the organ, acting as a biochemical factory for production of homeostatic proteins, hormones, enzymes and immunoglobulins for defense and reparative cells for tissue repair, organ regeneration, metabolism or other specialized functions. In mechano-dynamic organs such as the heart and lung, the endomural zones function to propel or exchange fluid or gas. The inner or endoluminal zone of organs may have functions similar to the endomural zone or act as yet another internal boundary or barrier layer.

In addition to solid or hollow organs with cavities, true tubular organs and organ components exist as vital body structures. Examples of tubular organs include the small intestine and the colon. Tubular organ components include major interpenetrating blood vessels in organs, e.g. the portal vein in the liver, the cavernous sinus in the brain. Examples of tubular tissue structures, include ducts, e.g. the bile duct, or blood vessels, e.g., arteries or veins.

Tubular organs and tissue structures in general have a laminated, multi-layer “tube-in-tube” structure made of at least three layers. All of these tubular organs, organ components or tissue structures may be characterized in similar fashion as outlined for organs above into ectoluminal, endomural and endoluminal zones. In tubular structures the ectoluminal zone may be characterized as the outer 10%±10 cross-sectional area, the endomural zone as the mid 80%±10 and the endoluminal zone as the inner 10%±10. Interestingly, tubular organs and tissue structures have defined histologic layers which generally correlate with these zones. The ectoluminal zone correlates with serosa or adventitia. The endoluminal zone correlates with the lamina propria, submucosa, muscularis, or media. The endoluminal zone correlates with the intima or mucosa.

Other solid organs may be viewed as containing two general regions, i.e., an ectomural zone and an endomural zone. The “ectomural” or outer zone typically functions to protect and contain the organ. The endomural zone of the organ is typically the functional or “business end,” of the organ, acting as a biochemical factory for production of homeostatic proteins, hormones, enzymes and immunoglobulins for defense and reparative cells for tissue repair, organ regeneration, metabolism or other specialized functions. If a solid organ is cut in cross-section the ectomural zone may be characterized as the outer approximately 20% of the cross-sectional area, and the endomural zone as the inner approximately 80% of the cross-sectional area.

II. Channels or Connecting Spaces

In some situations, it is necessary to create a “channel” or “connecting space” to alter tissue function. The terms “channel” and “connecting space” are used interchangeably herein. In one embodiment, the channel that may be hollow, filled, or lined with polymer or other structural material, an supplemental material or a combination thereof, or a device such as a catheter. These channels are used to connect spaces or convey materials from one space, or zone, to another. For example, the channel can be used to convey hormones produced in a gland such as the adrenal gland from the middle of the gland to the exterior. In another example, the channel is used to convey materials produced by the liver to a site on the exterior of the liver. In still another example, the channel is used to create a blood vessel equivalent to deliver blood from the middle of the myocardium to the ventricle.

In one embodiment, one or multiple layers or masses of a material are placed in the connecting space to isolate, cordon off or otherwise limit access from one zone, e.g., the endomural zone, to another zone, e.g. the endoluminal zone. These materials may be composed of organic or inorganic, solid, liquid or gas substances. They may be non-porous, or partially or selectively porous. These may include supplemental materials, such as pharmacologic agents, cells, tissue fragments, microorganisms, viral agents, or any other material which may be necessary to modify or isolate tissue function.

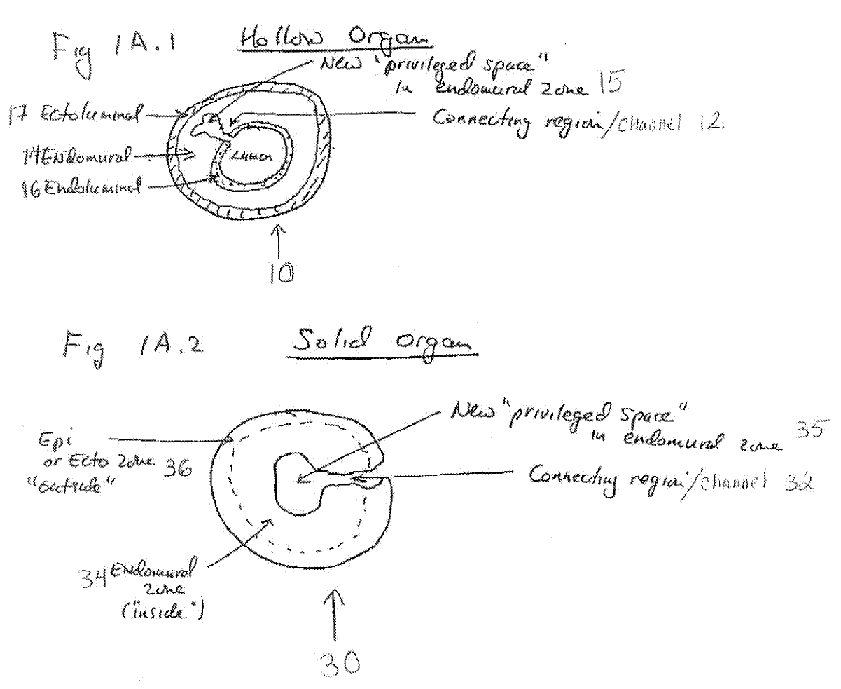

FIG. 1A.1 is a diagram of a hollow organ 10 showing a channel 12 leading from the endoluminal zone 16 to the endomural zone 14. A “privileged space” 15 has been created by removing tissue from the endomural zone. The ectoluminal zone is designated as element 17. FIG. 1A.2 is a diagram of a solid organ 30 showing a channel 32 leading from the ectomural zone 36 to the endomural zone 34. A “privileged space” 35 has been created by removing tissue from the endomural zone. FIGS. 1B-1D show delivery of supplemental materials in to various zones. FIG. 1B shows delivery of a material 20 at the tip of a device 18 through the connecting region 12 to the endomural zone 16. FIG. 1C shows delivery of a material 20 at the device 18 tip throughout the interior endomural zone 16 and channel or connecting region 12, i.e., within the endomural and endoluminal zones. FIG. 1D shows delivery of a material 20 throughout the endomural zone 16, connecting region 12, and exterior epi zone 22. Supplemental materials can be delivered, deposited and/or actuated either prior to, coincident with, following or continuously during the space making process.

b. Devices for Creating Channels

The channels can be created by removal of tissue or through insertion of a mechanical device such as a needle, catheter, stent or polymeric or tissue liner. Suitable devices for creating channels include a penetrator that is a sleeve shaped as a surgical needle, which may be fixed in place or have longitudinal mobility; a sharp pointed auger, of tubular structure, which can be disposed within the penetrator where it can have a restricted, controlled, longitudinal, relative movement which at rest or when gyrating; a laser, ultrasound, cryotherapy or radiofrequency means for creating a lumen; needles, catheters, and tubes. These may be formed of surgical steel or a polymeric material.

III. Supplemental Materials

As used herein “supplemental materials” means materials that act as supports and/or barriers and/or means for delivering agents that have a pharmacologic, biochemical or physiologic effect in the organ, organ component or tissue structure to which there are applied or at a site beyond. The supplemental material is typically applied to the site or region of interest in the form of a depot/reservoir, a continuous layer or a discontinuous layer.

A wide variety of supplemental materials can be placed in the channels, lumens, and/or privileged spaces. In one preferred embodiment the supplemental material acts as a barrier or support material. Suitable barrier or support materials include polymeric and non-polymeric materials, such as ceramics, glasses, or metals, or other inorganic materials. In another preferred embodiment, the supplemental material is a means for delivering agents that have a pharmacologic, biochemical or physiologic effects in vivo. Suitable supplemental materials include pharmacologic agents, cells, tissue fragments, microorganisms, viral agents, and other reagents modifying tissue function. In some embodiments, the supplemental material contains both supportive or barrier materials and one or more agents that have a pharmacologic, biochemical or physiologic effects in vivo.

In one preferred embodiment, the supplemental materials replace tissue function, suppress tissue function or enhance and/or augment tissue function or add additional or alternative tissue functions, which are not native to the tissue being treated with the supplemental material, either at the physiologic level, supra physiologic level, or hypophysiologic level or pharmacologic level.

The supplemental materials can be administered so that the material, or its effect, is observed at a distance into the tissue, i.e. not just on the edge of the space. This can be achieved not just through the use of the channel or connecting region, but through the use of permeation enhancers, dissolving or dispersing the enhancing or therapeutic agent in a solvent that penetrates the surrounding tissue, inclusion of surfactants, ultrasound, high or alternating pressure, vibration, electroporation, osmotic gradients or other transporting means.

In the broadest sense, the supplemental materials can include proteins (as defined herein, including peptides unless otherwise specified), saccharides, polysaccharides and carbohydrates, nucleic acids, and synthetic organic and inorganic materials.

The supplemental material can be in the form of a solid, or semi-solid material, a gas, or a liquid. The supplemental material may contain pharmacologic agents, cells, tissue fragments, microorganisms, or viral agents. For example, the supplemental material may replace tissue function, such as endothelial cells and/or smooth muscle cells, either adult, progenitor, embryonic or stem cells or admixtures thereof added for blood vessel formation, hepatocytes for liver replacement, or glandular cells for endocrine function. The supplemental materials may suppress tissue function, adding white blood cells for local tumor or abcess treatment. In another embodiment, the supplemental materials may enhance tissue function, for example, bone marrow may be used for extra-osseous hematopoeisis.

Any of the supplemental materials described herein can be mixed with other materials to improve their physiological compatibility. These materials include buffers, physiological salts, conventional thickeners or viscosity modifying agents, fillers such as silica and cellulosics, and other known additives of similar function, depending on the specific tissue to which the material is to be applied.

a. Barrier and/or Support Materials

The barrier and/or support materials may be polymeric or non-polymeric materials, such as ceramics, glasses, or metals, or other inorganic materials, or combinations thereof. In one embodiment, the supplemental material contains a mixture of polymeric and non-polymeric materials. In a preferred embodiment, supplemental materials that function as barriers or supports are used to isolate tissue zones, to line or pave newly created privileged zones or other tissue cavities, either native or spaces therapeutically created spaces or spaces containing therapeutics. These materials can be used in combination with, devices, stents, tissues, sensors or other added structures where the supplemental material is applied as an overcoating or coating in juxtaposition to the devices, stents, tissues, sensors or other added structures. The barrier or support materials can also be used to fix and affix exogenously applied bioactive agents.

Polymeric materials can be utilized within the channels or privileged spaces as reservoirs/depots for the delivery of therapeutic agents. Alternatively, the polymeric materials can be applied in the form of a layer that lines the channel or privileged space. The layer may be a continuous layer or a discontinuous layer.

Suitable polymeric materials include biocompatible polymeric materials (solids, gels, liquids, and mixed forms) in the form of single or multiple layer particles, spheres, polygonal prisms, capsules, stars, amorphous forms, and chopped fibers. The polymeric materials may be synthetic or natural and biodegradable or non-biodegradable.

Reservoirs/depots can also be formed from metal prisms (containers) filled with solid, gel, or liquid polymers with or without admixed bioactive/therapeutic agents.

a. Selection of Polymeric Materials

Representative synthetic polymers are: poly(hydroxy acids) such as poly(lactic acid), poly(glycolic acid), and poly(lactic acid-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terephthalates such as poly(ethylene terephthalte), polyvinyl alcohols, polyvinyl ethers, polyvinyl ester, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-coaprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxyacids such as lactic acid and glycolic acid, and copolymers with PEG, polyanhydrides, poly(ortho)esters, polyurethanes, poly(butyric acid), poly(valeric acid), poly(lactide-coaprolactone), blends and copolymers thereof.

Representative natural polymers include proteins, such as zein, modified zein, casin, gelatin, gluten, serum albumin, or collagen, and polysaccharides, such as cellulose, dextrans, hyaluronic acid, polymers of acrylic and methacrylic esters and alginic acid. These are not preferred due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses, acrylic or methacrylic esters of above natural polymers to introduce unsaturation into the biopolymers.

Examples of preferred non-biodegradable polymers include ethylene vinyl acetate, poly(meth)acrylic acid, polyamides, copolymers and mixtures thereof. In general, the biodegradable materials degrade either by enzymatic hydrolysis or exposure to water in vivo, or by surface or bulk erosion.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

These materials can be further categorized as follows.

### i. Materials Which Polymerize or Alter Viscosity as a Function of Temperature

Poly(oxyalkene) polymers and copolymers such as poly(ethylene oxide)-poly(propylene oxide) (PEO-PPO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described by U.S. Pat. Nos. 3,829,506; 3,535,307; 3,036,118; 2,979,578; 2,677,700; and 2,675,619, the teachings of which are incorporated herein. Polyoxyalkylene copolymers are sold by BASF and others under the tradename Pluronic™. Preferred materials include F-I27, F-108, and for mixtures with other gel materials, F-67. These materials are applied as viscous solutions at room temperature or lower which solidify at the higher body temperature. Another example is a low Tm and low Tg grade of styrene-butadiene-styrene block copolymer from Polymer Concept Technologies, C-flex™. Polymer solutions that are liquid at an elevated temperature but solid at body temperature can also be utilized. For example, thermosetting biodegradable polymers for in vivo use are described in U.S. Pat. No. 4,938,763 to Dunn, et al.

Several divalent ions including calcium, barium, magnesium, copper, and iron are normal constituents of the body tissues and blood. These ions can be used to ionically crosslink polymers such as the naturally occurring polymers collagen, fibrin, elastin, agarose, agar, polysaccharides such as hyaluronic acid, hyalobiuronic acid, heparin, cellulose, alginate, curdlan, chitin, and chitosan, and derivatives thereof cellulose acetate, carboxymethyl cellulose, hydroxymethyl cellulose, cellulose sulfate sodium salt, and ethylcellulose. Materials that can be crosslinked photochemically, with ultrasound or with radiation.

Materials that can be crosslinked using light, ultrasound or radiation will generally be those materials which contain a double bond or triple bond, preferably with an electron withdrawing substituent attached to the double or triple bond. Examples of suitable materials include the monomers which are polymerized into poly(acrylic acids) (i.e., Carbopols™), poly(acrylates), polyacrylamides, polyvinyl alcohols, acrylated polyethylene glycols, and ethylene vinyl acetates. Photopolymerization requires the presence of a photosensitizer, photoinitiator or both, any substance that either increases the rate of photoinitiated polymerization or shifts the wavelength at which polymerization occurs. The radiolysis of olefinic monomers results in the formation of cations, anions, and free radicals, all of which initiate chain polymerization, grafting and crosslinking and can be used to polymerize the same monomers as with photopolymerization. Photopolymerization can also be triggered by applying appropriate wavelength to a cyclo-dimerizable systems such as Coumarin and Cinnamic acid derivatives. Alpha-hydroxy acids backbone can be activated to carbonium ion. A COOH or SO₃H functionality can be inserted that can be subsequently reacted to amine containing ligands Materials that can be crosslinked by addition of covalent crosslinking agents such as glutaraldehyde.

Any amino containing polymer can be covalently crosslinked using a dialdehyde such as glutaraldehyde, or succindialdehyde. Examples of useful amino containing polymers include polypeptides and proteins such as albumin, and polyethyleneimine. Peptides having specialized function, as described below, can also be covalently bound to these materials, for example, using crosslinking agents, during polymerization.

Polymers with free carboxylic acid or other anionic groups (e.g., sulfonic acid), such as the acrylic acid polymers noted above, can be used alone or added to other polymeric formulations to enhance tissue adhesiveness. Alternatively, materials that have tissue binding properties can be added to or bound to the polymeric material. Peptides with tissue adhesion properties are discussed below. Lectins that can be covalently attached to a polymeric material to render it target specific to the mucin and mucosal cell layer could be used. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus adoratus, Lens culinaris, Limulus polyphemus, Lysopersicon eseulentum, Maclura pomifera, Momordica charantia, Mycoplasma gallisepticum, Naja mocambique, as well as the lectins Concanavalin A, Succinyl-Concanavalin A, Triticum vulgaris, Ulex europaeus, I, II and III, Sambucus nigra, Maackia amurensis, Limax fluvus, Homarus americanus, Cancer antennarius, and Lotus tetragonolobus.

The attachment of any positively charged ligand, such as polyethyleneimine, polylysine or chitosan to any microsphere or polymeric chain may improve bioadhesion due to the electrostatic attraction of the cationic groups to the net negative charge of the mucus. A surfactant-like molecule bearing positive charge and a hydrophobic core would be compatible with the bilayer membrane. This molecule will distribute its core and the positive charge to minimize energy of interaction and hence will be more tissue adhesive. The mucopolysaccharides and mucoproteins of the mucin layer, especially the sialic acid residues, are responsible for the negatively charged surface layer. Any ligand with a high binding affinity for mucin could also be covalently linked to the polymeric material.

Polymeric materials can also be used as tissue adhesives. In one form, fibrin is used. This has the advantage that it can be formed easily in situ using the patient's own fibrinogen, blood or serum, by addition of thrombin and calcium chloride. The materials described above can also be used. Other polymeric tissue adhesives that are commercially available include cyanoacrylate glues, GRF (Gelatin-resorcinol-formaldehyde) and polyethyleneglycol-poly(lactic acid and/or glycolic acid)-acrylates, both of which are applied as liquids and then photopolymerized.

The polymeric material can be designed to achieve a controlled permeability, either for control of materials within the cavity or into the tissue or for release of incorporated materials. There are basically three situations that the polymeric material is designed to achieve with respect to materials present in the lumen: wherein there is essentially passage of only nutrients (small molecular weight compounds) and gases from the lumen through the polymeric material to the tissue lumen surface; wherein there is passage of nutrients, gases and macromolecules, including large proteins and most peptides; and wherein there is passage of nutrients, gases, macromolecules and cells. The molecular weight ranges of these materials are known and can therefore be used to calculate the desired porosity. For example, a macromolecule can be defined as having a molecular weight of greater than 1000 daltons; cells generally range from 600-700 nm to 10 microns, with aggregates of 30-40 microns in size. For passage of cell, the material must possess or develop a macroporous structure.

### ii. Materials That Have Decreased Volume Following Polymerization

Under certain circumstances it may be useful to produce a polymer in situ which occupies a smaller volume than the solution from which it is applied, for example, as an adhesive for the cavity to hold the walls together. The polymerization can be accompanied by “syneresis” or expulsion of water from the polymer, during polymerization. Besides reducing mass of the product, this process may yield porous products that may be desirable for healing. Syneresis occurs when a polymerization reaction occurs with reaction of a large number of fractional groups per unit volume (high crosslinking density or when dilute solutions of reactants are polymerized and the amount of water in the formulation exceeds the intrinsic swelling capacity of the resulting polymer. The latter may occur, for example, when dilute solutions of PEG-diacrylate are polymerized (e.g., less than or equal to 5% macromer).

The polymeric material is designed to achieve a controlled permeability, either for control of materials within the lumen or for release of incorporated materials. There are basically three situations that the polymeric material is designed to achieve with respect to materials present in the lumen: wherein there is essentially passage of only nutrients (small molecular weight compounds) and gases from the lumen through the polymeric material to the tissue lumen surface; wherein there is passage of nutrients, gases and macromolecules, including proteins and most peptides; and wherein there is passage of nutrients, gases, macromolecules and cells. The molecular weight ranges of these materials are known and can therefore be used to calculate the desired porosity. For example, a macromolecule can be defined as having a molecular weight of greater than 1000 daltons; cells generally range from 600-700 nm to 10 microns, with aggregates of 30-40 microns in size.

### iii. Materials That Have Increased Volume Following Polymerization

Under certain circumstances it may be useful to produce a polymer in situ which occupies a larger volume than the solution from which it is applied, for example, as a volume expander or “tissue expander” to progressively create a new cavity to ease pressure or allow subsequent therapeutic application or natural ingress of fluid, gas or cells. Further swelling may act as a seporator preventing contact of tissue surfaces that would otherwise be proximate. As such swellable materials may be delivered or polymerized in situ. Materials may be hygroscopic, dessicated, dehydrated or admixtures thereof.

b. Materials Having a Pharmacologic, Biochemical or Physiologic Effect

Materials having a pharmacologic, biochemical or physiologic effect can be used as a supplemental material or can be incorporated into another supplemental material, such as a supplemental material that acts as a barrier or support.

In one embodiment, the supplemental material is administered to produce a reduced/hyponormal organ response. These supplemental materials include antibiotics, antiproliferatives, including drugs which act on microtubles or microfilaments (vinca alkaloids, taxol, taxanes, colchicine, discodermalide, epothilones, cytochalasins), alkylating agents, intercalating agents, transcription or translation inhibitors, topoisomerase inhibitors, anti-metabolites, and anti-mitotics, telomerase inhibitors, swivilase/gyrase inhibitors, modulators of cell-extracellular matrix interactions including cell growth inhibitors and anti-adhesion molecules, enzymes and enzyme inhibitor, immunosuppressants (sirolimus, tacrolimus, everolimus, pimecrolimus), antibodies and bioactive fragments thereof (including humanized, single chain, and chimeric antibodies; especially antibodies against growth factors such as platelet-derived growth factor (PDGF), fibroblast growth factor (FGF), epidermal growth factor (EGF), vascular endothelial growth factor (VEGF), transforming growth factor-beta (TGF-beta), scatter factor, human growth factor (HGF), and nerve growth factor (NGF), etc.), oligonucleotide drugs (including small interfering RNAs (siRNA), antisense, non-sense, aptamers, ribozymes, external guide sequences for ribonuclease P, and triplex forming agents), and anti-cell migratory agents.

In another embodiment, the supplemental material is administered to produce in an amplified/hypernormal organ response. These materials include growth factors, such as platelet-derived growth factor (PDGF), fibroblast growth factor (FGF), epidermal growth factor (EGF), vascular endothelial growth factor (VEGF), transforming growth factor-beta (TGF-beta), scatter factor, human growth factor (HGF), and nerve growth factor (NGF), etc.), extracellular matrix proteins, such as collagen, elastin, fibrin, fibrinogen, and vitronectin, the intermediate filament protein vimentin, C-reactive protein (CRP), hemoglobin, transferrin, insulin, thyroid hormone, adrenal corticotropic hormone (ACTH), aldosterone, angiotensin I, II, and serum.

Other supplemental materials that may be administered include anti-hypertensives, antivirals, antiinflammatories, both steroidal and non-steroidal, anti-spasmodics including channel blockers, anticoagulants, antigen and vaccine formulations, vasodilating agents, anti-thrombotic agents, thrombocytic agents, nitrates, lipid and cholesterol sequestrants and other agents which may modulate vessel tone, function, arteriosclerosis, and the healing response to vessel or organ injury post intervention.

In another embodiment, the supplemental material includes one or more cells for local delivery to defined regions of an organ or organ component. Cells may be incorporated into another supplemental material, such as a polymeric material, for example, as a suspension which forms a gel at the tissue surface that allows the cells to grow and in some cases to proliferate. The cells may be autologous, allogeneic, or xenogeneic. The cells can be living (whether naturally occurring or produced through recombinant DNA technology), artificial cells, cell ghosts (i.e., RBC or platelet ghosts), or pseudovirions, to serve any of several purposes. For example, the cells may be selected to produce specific agents such as growth factors at the local tissue location.

Cells administered as the supplemental material or incorporated into another supplemental material may also be stem cells, such as cord blood stem cells, adult stem cells (i.e. from bone marrow), or embryonic stem cells, or progenitor cells corresponding to the type of tissue at the treatment location or other cells providing therapeutic advantages. For example, liver cells might be incorporated into the polymeric material and implanted in a lumen created in the liver of a patient to facilitate regeneration and closure of that lumen. This might be an appropriate therapy in cases where diseases (e.g. cirrhosis, fibrosis, cystic disease or malignancy) results in non-functional tissue, scar formation or tissue replacement with cancerous cells. Similar methods may be applied to other organs as well.

Cells incorporated may be either similar or dissimilar to those natively resident in the organ or organ component. As such, in the case of dissimilar cells, the organ shell may be simply acting as a “rental space,” i.e., providing a domicile for otherwise foreign cells to remain resident or engraft or otherwise take up residence in a more stable environment. As such these differing cells may then provide their normal function, albeit in an atypical residence. Alternatively these cells, by functioning in an alien location, may provide alternative function, i.e. hypernormal or hyponormal function.

### i. Incorporation and Release of Materials Having a Pharmacologic, Biochemical or Physiologic Effect in Another Supplemental Material

Materials having a pharmacologic, biochemical or physiologic effect (also referred to herein as “bioactive agents”) can be physically incorporated or chemically incorporated into another supplemental material, such as a polymeric material. Release of a physically incorporated bioactive agent is achieved by diffusion and/or degradation of the polymeric material; release of a chemically incorporated bioactive agent is achieved by degradation of the polymer or of a chemical link coupling the peptide to the polymer, for example, a peptide which is cleaved in vivo by an enzyme such as trypsin, thrombin or collagenase. Similarly any other chemical linkage may be degraded via contained means or external means to “free” and release the bioactive agents. In some cases, it may be desirable for the bioactive agent to remain associated with the polymeric material permanently or for an extended period, until after the polymeric material has degraded and removed from the site.

In most cases, it is possible to physically incorporate a bioactive agent by mixing with another supplemental material, such as a monomer or crosslinkable polymer, prior to application to the tissue surface and polymerization. The material can be mixed into a monomer solution to form a solution, suspension or dispersion. In one embodiment, a bioactive agent can be encapsulated within delivery devices such as microspheres, microcapsules, liposomes, cell ghosts or pseudovirions, which in themselves effect release rates and uptake by cells such as phagocytic cells. Additionally a bioactive agent may be added at the time of polymeric in situ formation or surface or cavity polymerization.

### Chemical Coupling of Bioactive Agents to Other Supplemental Materials

Bioactive agents can be chemically coupled to another supplemental material, such as polymeric material, before or at the time of polymerization. In the preferred embodiment, the bioactive agents are chemically coupled prior to administration of the polymeric to the tissue or organ. Several polymeric biocompatible materials are amenable to surface modification in which surface bound bioactive molecules/ligands exhibit cellular binding properties. These methods are described by Tay, Merrill, Salzman and Lindon in Biomaterials 10 11-15 (1989).

Covalent linkages can be formed by reacting the anhydride or acid halide form of an N-protected amino acid, poly(amino acid) (two to ten amino acids), peptide (greater than 10 to 100 amino acids), or protein with a hydroxyl, thiol, or amine group on a polymer. The amine groups on the amino acid or peptide must be protected before forming the acid halide or anhydride, to prevent self-condensation. N-protection is well known by those skilled in the art, and can be accomplished by use of various protecting groups, such as a carbobenzoxy (CBZ) group.

The term “protecting group” as used herein refers to a moiety which blocks a functional group from reaction, and which is cleavable when there is no longer a need to protect the functional group. Examples of functional groups include, but are not limited to, amino, hydroxy, thio, and carboxylate groups. Examples of protecting groups are well known to those skilled in the art.

A carboxylate-containing compound can contain various functional groups, such as hydroxy, thio, and amino groups, that can react with an acid halide or anhydride. These functional groups must be protected before forming an acid chloride or anhydride to avoid self-condensation. After formation of the acid chloride or anhydride, and subsequent reaction with the hydroxyl, thiol, or amino group(s) on another molecule, the protecting group can be removed in a “deprotecting” step. The N-protected amino groups can be deprotected by means known to those skilled in the art. Any hydroxy or thio groups on these compounds must be protected so as not to react with the acid halides or anhydrides. Examples of suitable protecting groups for alcohols include but are not limited to trialkyl silyl groups, benzyl ethers, and tetrahydropyranyl ethers. These groups can be protected by means known to those skilled in the art, and can be subsequently deprotected after the esterification is complete. Examples of protecting groups can be found in Greene, T. W., and Wuts., P; G. M., “Protective Groups in Organic Synthesis 2d Ed., John Wiley & Sons, Inc., pp. 317-318 (1991).

A non-limiting method for preparation of acid halide derivatives is to react the carboxylic acid with thionyl chloride, preferably in benzene or toluene with a catalytic amount of DMF. A known method for producing anhydrides is to react the carboxylic acid with acetic anhydride. In this reaction, as acetic acid is formed, it is distilled out of the reaction vessel. Peptides can be covalently bound to the polymeric material, for example, when the polymeric material is a polymer of an alpha hydroxy acid such as poly(lactic acid), by protecting the amine functionality on the peptide, forming an acid halide or anhydride of the acid portion of the polymer, reacting the acid halide or anhydride with free hydroxy, thiol, or amine groups on the polymer, then deprotecting the amine groups on the peptide to yield polymer having peptide bound thereto via esterification, thioesterification, or amidation. The peptide can also be bound to the polymer via a free amine using reductive amination with a dialdehyde such as glutaraldehyde.

The ester groups on a polyester surface can be hydrolyzed to give active hydroxy and carboxyl groups. These groups can be used to couple bioactive molecules. Preferably, before converting the active carboxylate group to the acid halide or anhydride form, the active hydroxy group is protected to avoid reaction with the resulting acid halide or anhydride. As a non-limiting example, the active hydroxy group can be protected as a benzyl ether. The active carboxyl group can then be converted to the acid halide or anhydride, and reacted with a hydroxy or amino group on a second compound to form an ester or amide linkage. The O-protected hydroxy group can then be deprotected.

Polyanhydrides can be partially hydrolyzed to provide carboxyl groups. The resulting carboxyl groups can be converted to acid halides, which can be reacted with amino acids, peptides, or other amine containing compounds with binding properties and form an amide linkage.

Polyesters and polylactones can be partially hydrolyzed to free hydroxyl and carboxyl groups. The hydroxyl groups can be protected by means known to those skilled in the art, and the carboxyl groups converted to acid halides. The acid halides can be reacted with amino acids, peptides, or other amine containing compounds with binding properties and form an amide linkage. Alternatively, if the hydroxyl groups are primary or secondary hydroxyl groups, they can be oxidized to aldehydes or ketones, and reacted with amines via reductive amination to form a covalent linkage.

Polyamides can be partially hydrolyzed to provide free amine and carboxylic acid groups. The amine group can then be reacted with an amino acid or peptide in which the amine groups have been protected, and the carboxyl groups have been converted to acid halides. Alternatively, the amine groups on the polyamide can be protected, and the carboxyl groups converted to acid halides. The resulting acid halides can then be reacted directly with the amine groups on amino acids or peptides.

Polyalcohols with terminal hydroxy groups can be appended with amino acids or peptides. One first protects the amine groups, then converts the carboxyl groups on the amino acid or peptide to acid halides. The acid halide can be reacted directly with the hydroxy group to provide an ester linkage.

The acid halides described above can also be reacted with thiol groups to form thioesters.

IV. Application of the Supplemental Materials

In one embodiment, the supplemental material is a biocompatible polymeric material having a variable degree of fluency in response to a stimulus or mechanical pressure. The material is such that it is substantially non-fluent in vivo upon completion of the coating process. The material, in its fluent form or a conformable form, is positioned in contact with a tissue or device surface to be coated and then stimulated to render it non-fluent or conformed. The polymeric material is applied to the channel, cavity or endomural void using catheters, syringes, or other percutaneous or surgical applicators, sprays, depending on the tissue surface or device to which it is applied, using the devices described above or devices known to those skilled in the art. The supplemental material is typically applied to the site or region of interest in the form of a depot/reservoir, a continuous layer or a discontinuous layer. In one embodiment, the applied layers may be discontinuous to allow interchange of solutes, proteins, hormones, chemical messengers or cell “traffic.” Depending upon the porosity of the layer discontinuities, any or all of these materials may be exchanged.

The supplemental material typically will be applied to endoluminal, endomural, or ectoluminal regions or surfaces of organs, organ components, or tissue structures with hollow or tubular geometry, for example blood vessels, gut, and bladder or of “solid” organs that possess true spaces such as cavities, cavernous sinuses, lumens, etc., such as the heart, liver, kidney and pancreas. For solid organs that contain two general regions, i.e., an ectomural zone and an endomural zone, the supplemental typically will be applied to the ectomural or endomural regions or surfaces of these organs, such as to a channel or endomural void.

The supplemental materials are preferably applied using a single catheter or similar device with single or multiple lumens. The catheter should be of relatively low cross-sectional area. A long thin tubular catheter manipulated using endoscopic guidance is preferred for providing access to the interior of organ areas. Alternatively the device may have direct vision capabilities via contained fiberoptics or actual tip cameras (CCD, C-MOS, etc) or via echo sensing, ultrasound (US) sensing, Magnetic Resonance Imaging (MRI) or global positioning systems (GPS).

### Formation of Discontinuous Layer of Supplemental Material

Alternatively the supplemental materials so that a discontinuous layer is formed. In one embodiment, the materials that form the supplemental material are applied in a discontinuous fashion. This may occur via application of a preformed layer with preformed or otherwise contained discontinuities. For example, a dessicated or partially polymerized or asymmetrically adhesive layer (e.g., where one side is non-tacky, and the other side is tacky) into which gaps or spaces of identical or varying porosities or shapes exist. Alternatively the material may be applied in a fashion to create discontinuities. For example, via intermittent spraying as an application means (e.g. spray tip catheter, endoscope or trochar) is traversed or otherwise pulled across a tissue surface. Or via pulsed jetting, atomization, spray drying, sputtering, vapor deposition or other discontinuous or otherwise interrupted means. Alternatively the material may be applied in a continuous or discontinuous fashion, where the material further contains a “drop-out” material, which dissolves away, or is removed following application. For example if a molten polymer layer that contains a “drop out” material, such as a dissolvable particulate material or a material that will biodegrade or may be activated to disperse or otherwise disappear, is applied via a catheter with an application tip (i.e. nozzle system), then following application the “drop out” material will be removed from the polymer layer, leaving a discontinuous polymer layer. Examples of dissolvable materials include crystalline or particulate salts, sugars, proteins or any other dissolvable, degradable or activatable material, including materials which by virtue of the particulate size and shape create voids. Alternatively a discontinuous layer may be created by applying a first priming layer or a pattern layer in a discontinuous physical way (e.g. intermittent spray coating) followed by chemically or adhesively reacting a second applied material or layer (applied in either a physically continuous or discontinuous means) so that the net chemical or adhesive reaction between the two applications only occurs where both are in intimate contact. As such by virtue of one layer being discontinuous, the product of the reaction or adhesion of both materials will be a discontinuous layer, which is located only where both materials are in contact or reactive with each other.

### Application of Polymeric Supplemental Materials

Application of a polymeric material may be accomplished by extruding a solution, dispersion, or suspension of monomers, polymers, macromers, or combinations thereof through a catheter to coat or fill a tissue surface or cavity, then controlling formation of the coating by introducing crosslinking agents, gelling agents or crosslinking catalysts together with the fluent material and then altering the conditions such that crosslinking and/or gelling occurs. Thus, when a balloon catheter is used, a flow of heated or chilled fluid into the balloon can alter the local temperature to a level at which gelling or cross-linking is induced, thereby rendering the material non-fluent. Localized heating or cooling can be enhanced by providing a flow of heated or chilled liquid directly onto the treatment site. Thermal control can also be provided, however, using a fluid flow through or into the balloon, or using a partially perforated balloon such that temperature control fluid passes through the balloon into the lumen. Thermal control can also be provided using electrical resistance heating via a wire running along the length of the catheter body in contact with resistive heating elements. This type of heating element can make use of DC or radio frequency (RF) current or external RF or microwave radiation. Other methods of achieving temperature control can also be used, including light-induced heating using an internal optical fiber (naked or lensed). Alternatively as self-contained fluid flow system allowing inflow and outflow of fluids to the balloon, actuator or other material applying tip of surface may control polymer flow, melt, setup and cooling and fixation. The polymer formulation can contain components which convert light into heat energy. Similar devices can be used for application of light, ultrasound, or irradiation.

Alternatively the polymers may be delivered as solid materials of various configurations, e.g. rods, spheres, folded sheets, yarns, meshes, twines, ropes, particles, amorphous shapes, flakes, etc. Similarly hydrogel materials may be delivered with the above physical geometries in either the hydrated, partially hydrated or desiccated form. Further defined hydrogel shapes such as spikes, spheres with wicks and other tract and void shapes may be delivered for the purpose of void sealing or plugging or repair.

The process of fixing the shape of the polymeric material can be accomplished in several ways, depending on the character of the original polymeric material. For example, a partially polymerized material can be expanded using a balloon after which the conditions are adjusted such that polymerization can be completed, e.g., by increasing the local temperature or providing UV or visible radiation through an optical fiber. A temperature increase might also be used to soften a fully polymerized sleeve to allow expansion and facile reconfiguration and local molding, after which it would “freeze” in the expanded position when the head source is removed. Of course, if the polymeric sleeve is a plastic material which will permanently deform upon stretching (e.g., polyethylene, polyethylene terephthalate, nylon or polyvinyl chloride), no special fixation procedure is required.

IV. Medical Indications for Treatment

The supplemental materials are locally provided to alter, retard, or enhance function of an organ, organ component or tissue structure in response to changes in the function of an organ or organ component during the course of a lifetime. The supplemental materials may act locally, and/or may have effects at sites and regions distant to the site of administration. Organs that may be treated include those organized with endoluminal, endomural, or ectoluminal regions and surfaces, such as those with hollow or tubular geometry, including blood vessels, gut and bladder; “solid” organs, such as the heart, liver, kidney and pancreas, which possess true spaces such as cavities, cavernous sinuses, lumens, etc.; and solid organs containing two general regions, i.e., an ectomural zone and an endomural zone.

In one embodiment, the supplemental material may be applied to a vulnerable plaque in an atherosclerotic artery, preferably the supplemental material is used to treat and/or stabilize a vulnerable plaque or aneurysm.

Changes in organ function that may be treated application of the supplemental material include attainment of normal function, loss of function (hypo-normal function), enhancement of function (hyper- or supra-normal function). Hypo-normal function may develop due to atrophy, toxemia, environmental exposure, infection, inflammation, malignancy, injury, ischemia, malnutrition, radiation exposure, temperature alteration, infiltrative processes, fibrotic processes, calcification, lipid insulation, atherosclerosis, and physical and mechanical stressors. Hypernormal function may develop due to hyperplasia, hypertrophy, neoplasia, different types of stimulation, including nutritional, metabolic, and supplement-stimulation, cellular infiltrative processes, exposure to a number of factors, including environment, radiation, hormones, temperature and pharmacological exposure, hyperemia, hyper- or super-fusion, malignancy, physical and mechanical stressors, and tissue implantation or transplantation.

The supplemental material may be applied in an “organ shell,” i.e. an organ with a new privileged space, with remaining native parenchyma surrounding, to create a “hybrid organ.” As generally used herein a “hybrid organ” is an organ in which the original organ serves as a basic stromal housing or “rental space,” to provide basic essential functions for the new cells or tissues. Specifically the organ shell may provide any, some or all of these functions: containment, arterial blood supply, venous drainage, etc. For example a shelled out spleen may be injected or treated with liver cells in the privileged zone to create a hybrid spleen which functions as a neo-liver or the shelled out spleen could be injected or treated with bone marrow in the privileged zone to create a hybrid spleen with neo-marrow. Such hybrid organs may provide native or alternative tissue function.

In one embodiment a discontinuous layer of the supplemental material is added to an angioplasty, atherectomy, stent, or surgical site before, during, or after, the procedure.

Those skilled in the art will recognize, or be able to ascertain using no more than routine experimentation, many equivalents to the specific embodiments of the invention described herein. Such equivalents are intended to be encompassed by the following claims.

I claim:

1. A method of treating diseases, aging, trauma, environmental exposure, infection and other events or agents that alter tissue function in an organ, organ component or tissue structure comprising
creating a channel extending from the ectoluminal or ectomural zone to the endomural zone or creating a channel extending from the endoluminal zone to the endomural zone,
creating a privileged space in the endomural zone of the organ, organ component or tissue structure,
administering a supplemental material into the privileged space or the channel in an effective amount to continue normal native tissue function, suppress native tissue function, enhance native tissue function, or provide alternative tissue function.

2. The method of claim 1, comprising administering the supplemental material into the channel.

3. The method of claim 1 wherein the channel, privileged space, or a combination thereof is created with a device selected from the group consisting of ultrasound, cryotherapy or radiofrequency means for creating a lumen; a penetrator; an auger; a laser; a needle; catheters; and tubular members.

4. The method of claim 3, wherein the catheters or tubular members comprise balloons and means for tissue removal.

5. The method of claim 1, wherein the supplemental material comprises a polymeric material.

6. The method of claim 1, wherein the supplemental material comprises at least one bioactive agent.

7. The method of claim 6 wherein the bioactive agent is selected from the group consisting of pharmacologic agents, cells, tissue fragments, microorganisms, and viral agents.

8. The method of claim 7 further comprising delivering the bioactive agent beyond the site of administration, and applying means for enhancing permeation selected from the group consisting of permeation enhancers, solvents that penetrate tissue, surfactants, ultrasound, high or alternating pressure, vibration, electroporation, and osmotic gradients.

9. The method of claim 1 wherein supplemental material is used to isolate tissue zones, to line or pave newly created privileged zones or other tissue cavities, either native or spaces therapeutically created spaces or spaces containing therapeutics.

10. The method of claim 5 further comprising devices, stents, tissues, sensors, or other added structures, wherein supplemental material is applied as an overcoating or coating on the devices, stents, tissues, sensors, or other added structures.

11. The method of claim 1 wherein supplemental material comprises bioactive agents and a polymeric material, and wherein the polymeric material fixes or affixes the bioactive agents within the channel or privileged space.

12. The method of claim 1, wherein the supplemental material is applied either prior to, coincident with, following, or continuously during formation of the channel or privileged space.

13. A system for treating diseases, aging, trauma, environmental exposure, infection and other events or agents that alter tissue function in an organ, organ component or tissue structure, the system comprising
a first device for creating a channel extending from the ectoluminal or ectomural zone to the endomural zone,
a second device for creating a privileged space in the endomural zone of the organ, organ component or tissue structure, and
a third device for administering a supplemental material into the privileged space or the channel, wherein the first, second, and third devices may be the same device or different devices, and
the supplemental material.

14. A method for the therapeutic treatment of an organ, organ component or tissue structure in an animal, comprising applying a supplemental material comprising a bioactive agent to one or more regions of the organ, organ component or tissue structure.

15. The method of claim 14, wherein the supplemental material forms a discontinuous layer at the site of application.

16. The method of claim 14, wherein the supplemental material forms a depot or reservoir at the site of application.

17. The method of claim 14 wherein the supplemental material comprises a polymeric material selected from the group consisting of biodegradable synthetic polymeric materials, biodegradable natural polymeric materials and non-biodegradable polymeric materials.

18. The method of claim 14 wherein the bioactive agent is administered in an effective amount to produce a reduced or hyponormal organ response.

19. The method of claim 14 wherein the bioactive agent is administered in an effective amount to produce an amplified or hypernormal organ response.

20. The method of claim 14 wherein the supplemental material comprises one or more cells.

21. The method of claim 20 wherein the cells are stem cells.

22. The method of claim 14 wherein the organ or organ component is a pre-angioplasty, atherectomy, stent, or surgical site.

23. The method of claim 14 wherein the organ or organ component is a post-angioplasty, atherectomy, stent, or surgical site.

24. The method of claim 14, wherein the supplemental material is applied to a vulnerable plaque or aneurysm in an effective amount to treat and/or stabilize a vulnerable plaque or aneurysm.
